(19) United States
(12) Patent Application Publication
(10) Pub. No.: US 2020/0030570 A1
(43) Pub. Date: Jan. 30, 2020

(54) ELECTRONIC STRESS RELIEVING DEVICE

(71) Applicant: William KROSSNER, Clive, IA (US)

(72) Inventor: William KROSSNER, Clive, IA (US)

(21) Appl. No.: 16/519,536

(22) Filed: Jul. 23, 2019

Related U.S. Application Data
(60) Provisional application No. 62/702,376, filed on Jul. 24, 2018.

(51) Int. Cl. A61M 21/02; F21V 3/04; F21V 23/00

(52) U.S. Cl. CPC A61M 21/02; F21Y 2113/13; F21V 23/003; F21V 3/049

(57) ABSTRACT

An electronic stress relieving device has a base with at least one light source attached to the base. The at least one light source produces a light that passes through a light diffuser which is at least partially translucent and is disposed over the at least one light source so that the light is diffused when viewed through the light diffuser. An energy source is provided and electrically connected to the at least one light source and an electronic controller. The electronic controller allows a user to operate the at least one light source in a selected mode of operation and the user then views the pattern of light as it is displayed on the light diffuser to help relieve stress.

### CROSS REFERENCE TO RELATED APPLICATIONS

This application claims priority and herein incorporates by reference U.S. provisional patent application 62/702,376, filed Jul. 24, 2018.

### BACKGROUND OF THE INVENTION

Recent research indicates that stress levels have increased in the last thirty years. Stress is also associated with a greater risk of many diseases including high blood pressure, heart disease, obesity and diabetes. Stress may also be associated with the increase in the suicide rate which is reported to be up in recent years.

The causes of stress are varied and include things such as employment issues, financial worries, illness and death, moving and many others. Stressors are not easily changed, and in general, doctors and other health professionals recommend finding coping mechanisms that help the person manage stress. Often medication is prescribed, but these can have adverse side effects. Dietary guidelines can also be employed such as eating a healthy diet, avoiding caffeine, etc.

There is a need for a medication free treatment for helping an individual cope with stress that is easy, safe, economical to use and without the need to reference philosophical or other historical traditions to help manage stress.

### SUMMARY OF THE INVENTION

An electronic stress relieving device has a base with at least one light source attached to the base. The at least one light source produces a light that passes through a light diffuser which is at least partially translucent and is disposed over the at least one light source so that the light is diffused when viewed through the light diffuser. An energy source is provided and electrically connected to the at least one light source and an electronic controller. The electronic controller allows a user to operate the at least one light source in a selected mode of operation and the user then views the pattern of light as it is displayed on the light diffuser to help relieve stress.

Other features and advantages of the instant invention will become apparent from the following description of the invention which refers to the accompanying drawings.

### BRIEF DESCRIPTION OF THE DRAWINGS

FIG. 1 is a top view of an electronic stress relieving device according to an embodiment of the present invention.

FIG. 2 is a side view of the electronic stress relieving device shown in FIG. 1.

FIG. 3 is a side view of an electronic stress relieving device according to an embodiment of the present invention.

FIG. 4 is a top view of the electronic stress relieving device shown in FIG. 1 having three LED lights.

FIG. 5 is a top view of the electronic stress relieving device shown in figure one having a microprocessor.

FIG. 6 is a side view of the electronic stress reliving device shown in FIG. 3 having an alternative light diffractor.

FIG. 7 is a side view of an electronic stress reliving device having a base containing electronic components.

FIG. 8 is a top view of an electronic stress relieving device having only a light diffractor according to an embodiment of the present invention.

FIG. 9 is a top view of an electronic stress relieving device having only a light diffusor according to an embodiment of the present invention.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

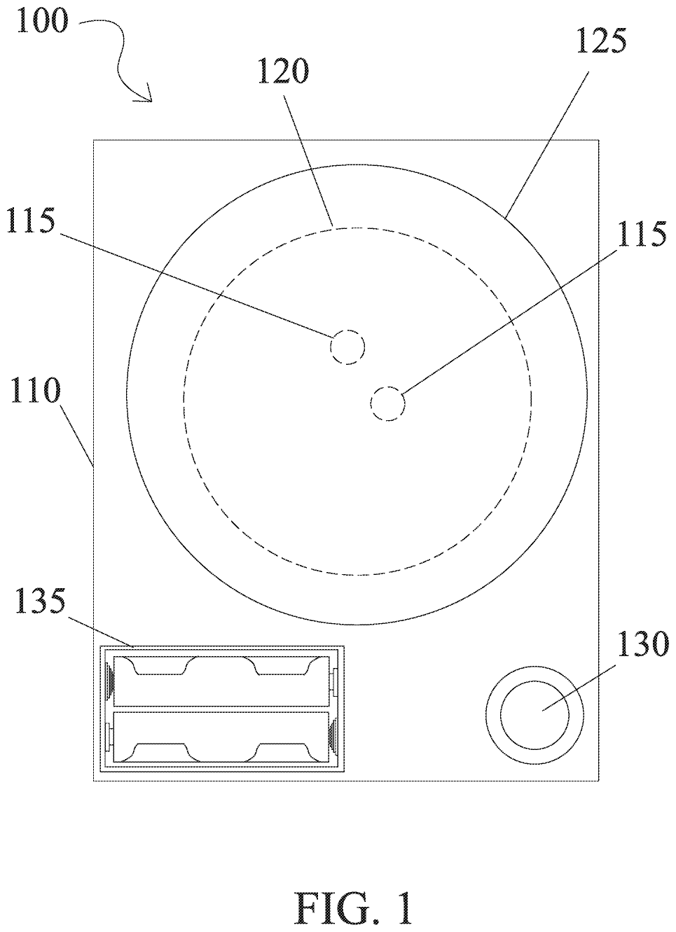

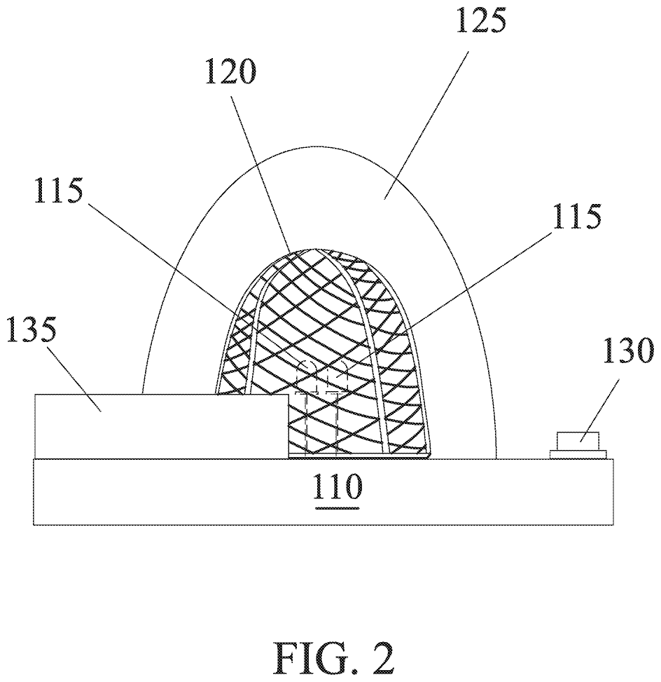

Referring to FIGS. 1 and 2, an electronic stress reliving device 100 is shown having a base 110 that is used to hold at least one LED (light emitting diode) 115 and a light diffractor 120 surrounded by a light diffuser 125. A battery 135 is used to provide the energy required to energize LEDs 115. A switch 130 is used to control electronic stress reliving device 100 as is known in the art. Of course, an AC adapter (not shown) may be provided to allow a user to plug electronic stress relieving device 100 into a wall outlet.

Light diffractor 120 is made of glass and serves to diffract the light produced by LEDs 115. The diffraction causes the light emitted to bend and reflect at different angles and lines rather than just points of light as naturally emitted by LEDs 115. Various angles and curves may be formed in the glass to provide facets that diffract the light. Of course other materials than glass such as plastic can be used but the diffractor should spread out the light. LEDs 115 are tri-color LEDs that emit red, green and blue light and cycle through all three colors when energized. Again, other LEDs may be used including, but not limited to single color LEDs. It should be understood, that LEDs are the light source of choice due to their low power requirements and low heat output, other light sources could be used such as small incandescent or florescent light sources could be used.

Light diffuser 125 softens the light from LEDs I 15 and spreads the light out to provide a visually pleasing and relaxing interplay of the colored light projected against light diffuser 125. Light diffuser 125 is a translucent plastic. Of course other materials may be used for light diffuser 125 as long as it is translucent and allows the user to view the light. Light diffuser 125 is a translucent white plastic, but other translucent colors may be used or may even be a clear translucent plastic.

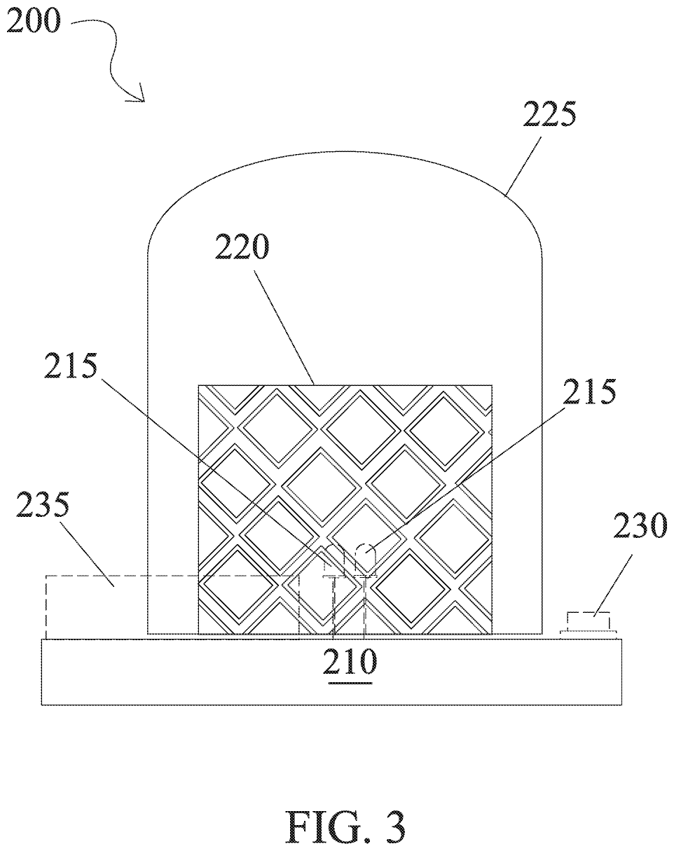

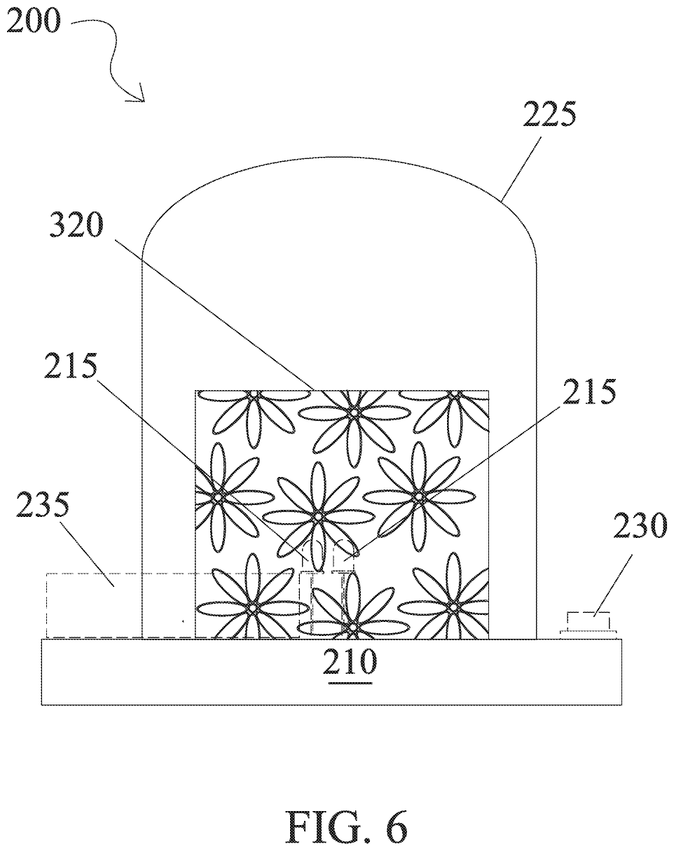

Now referring to FIGS. 3 and 6, an electronic stress relieving device 200 is shown having a base 210, battery 235, LEDs 215 and a switch 230 analogous as described with respect to FIGS. 1 and 2 above. In this embodiment, a light diffractor 220 (FIGS. 3) and 320 (FIG. 6) has a different geometry from the one shown in figure I. Because the geometry is different, the light emitted from LEDs 215 is diffracted in different ways to provide visually pleasing and varied light outputs. Of course many different geometries may be used and it may be desirable to provide the user with multiple diffractors to allow the user to choose various patterns to enhance use. A light diffuser 225 may also have a different geometry chosen to enhance the user's experience.

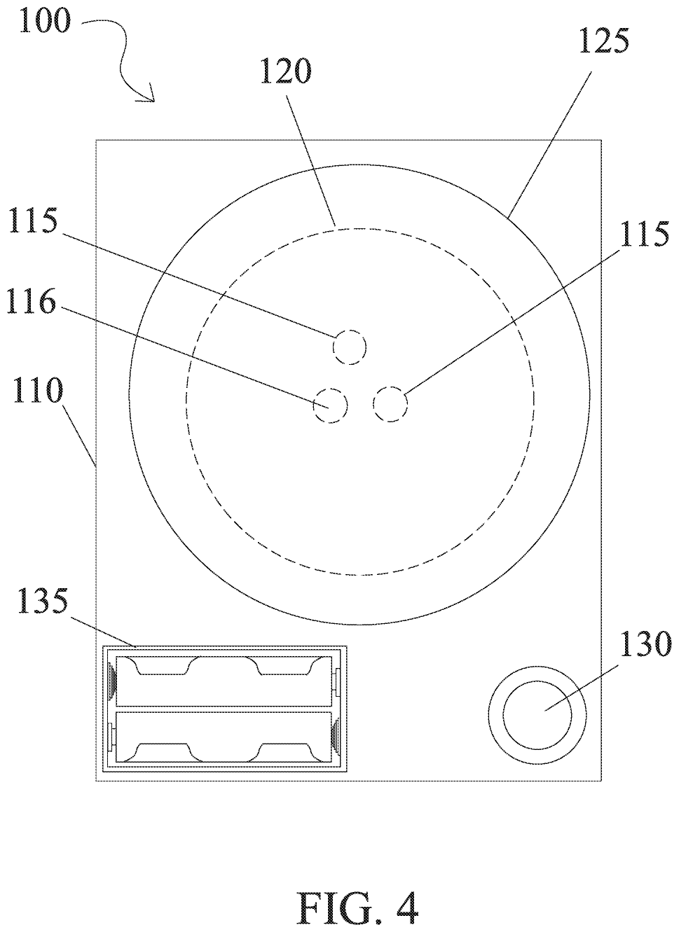

Referring now to FIG. 4, electronic stress relieving device 100 is shown having an additional LED 116. Of course any number of LEDs may be used although only three are shown. additionally, other light sources may be used as long as the power requirements are supplied as is known in the art.

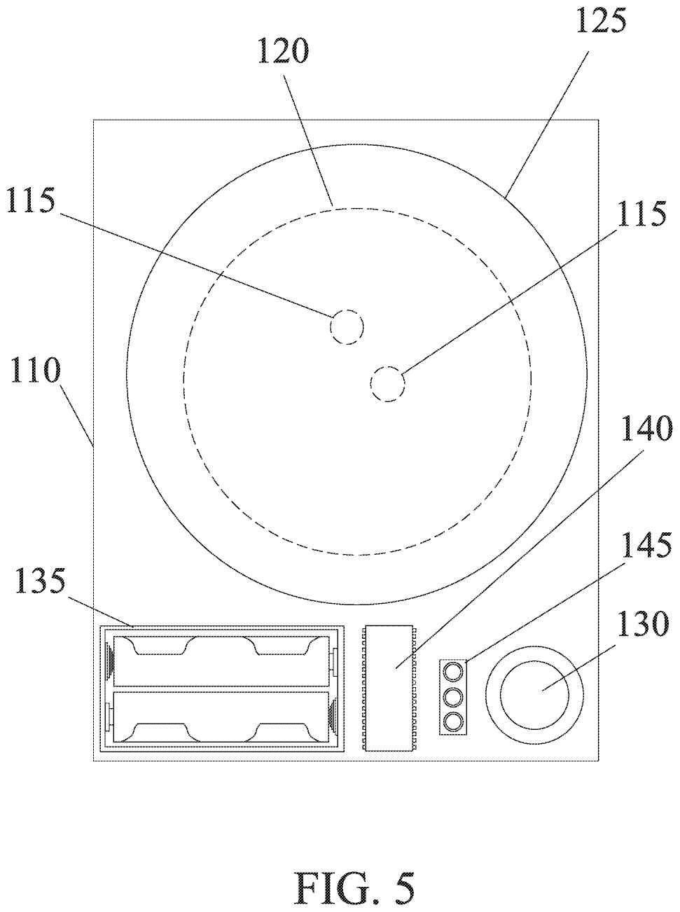

Referring to FIG. 5, a microprocessor 140 is used to further enhance the user's control over how the light is mixed since microprocessor 140 controls the color output rather than just cycling through the three colors as described with respect to FIG. 1 above. Colors can be made to flash or persist as is known in the art as well as controlling the intensity of the light. A control 145 is used to allow the user to select and control the light patterns selectable through microprocessor 140. Control 145 may be a plurality of switches or could be a slide switch or even a single switch that allows a user to cycle through the choices by repeated activation.

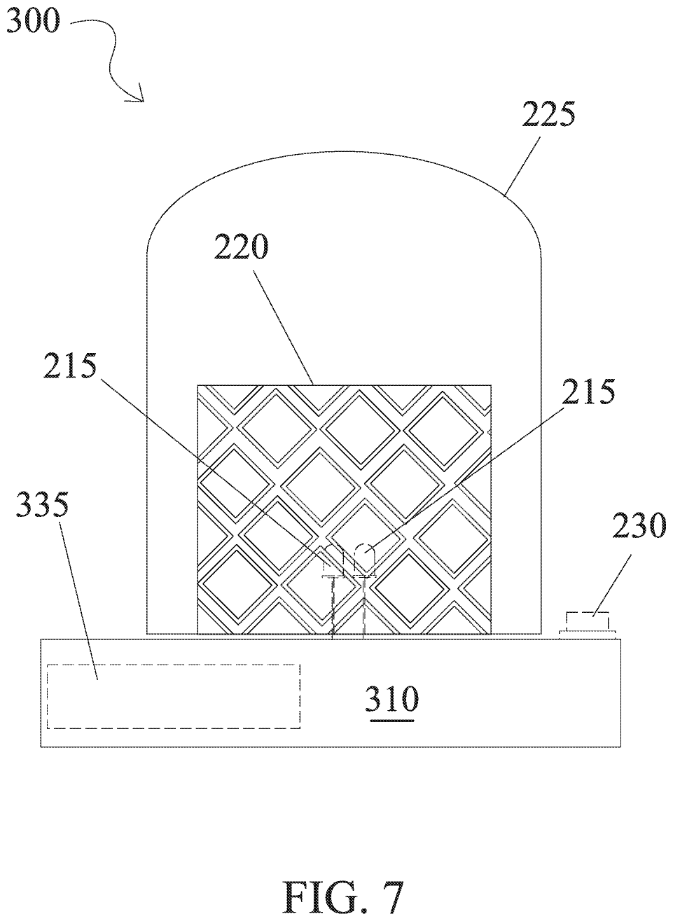

Now referring to FIG. 7, an electronic stress relieving device 300 is shown having a base 310 that includes a battery 335 and associated electronics (not shown) built in. Battery 335 is accessible using a removable panel (not shown) as is known in the art.

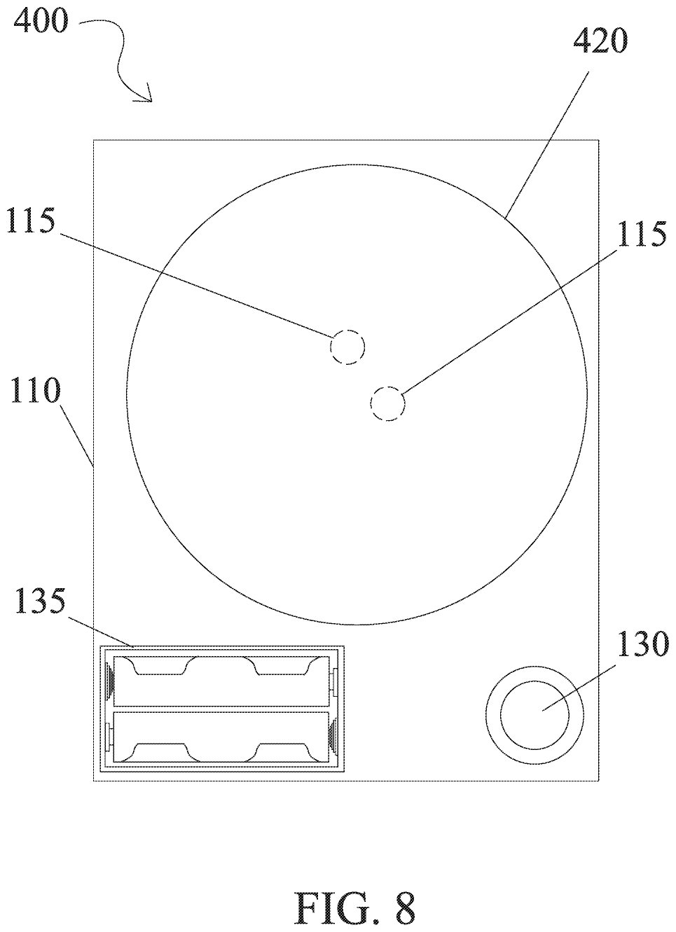

Referring now to FIG. 8, an electronic stress relieving device 400 is shown having a light diffractor 420 only on a base 410. In this embodiment, the diffuser is left off while still providing interesting diffraction patterns when the LED light is viewed through light diffractor 420. It would be possible to only include a light diffuser as well.

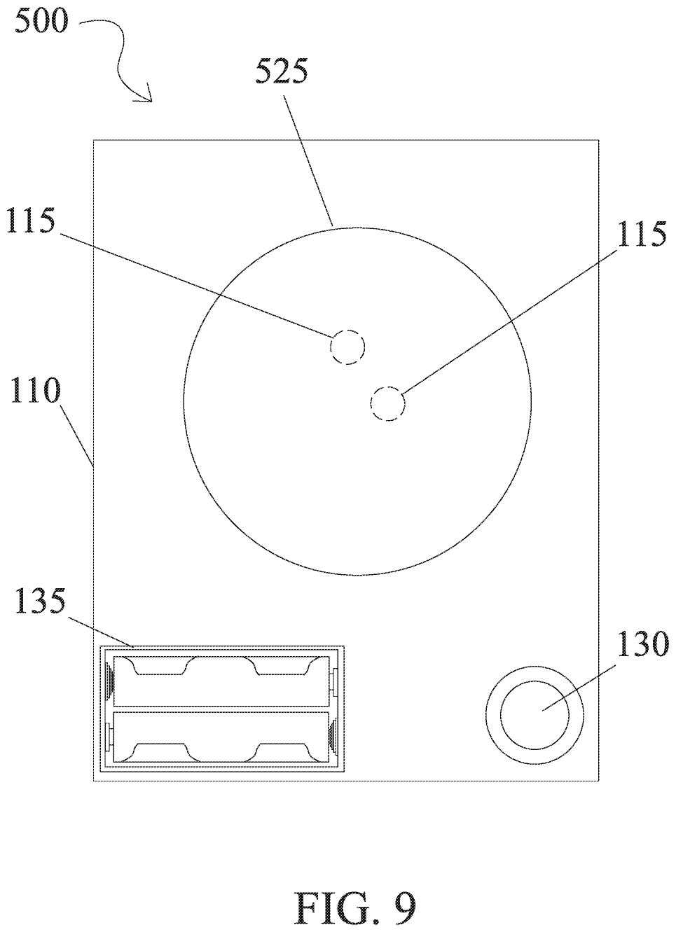

Now referring to FIG. 9, an electronic stress relieving device 500 is shown having a light diffusor 425 only on a base 510. In this embodiment, the diffractor is left off so that the user only sees a diffuse pattern of light that varies in intensity without the presence of a diffractor.

In use, the user places the device in a room, preferably dimly lit and without distractions and watches the changing patterns of light as they vary. The time the users spends observing the light can vary from a few minutes a day to longer periods several times a day. The power requirements of the LEDs vary from 1.5 to 4.5 volts. Typically, the power is supplied by batteries 135, 235 and 335 which can be one to three AA or AAA type batteries. Of course an adapter may be provided to allow the device to be powered by plugging it in to an outside source of power such as an electrical outlet. Additionally it is possible to provide a hand crank unit that allows the user to provide the energy needed using a hand charging means (not shown) as is known in the art.

The power consumption of the LEDs is very low. When a microprocessor is used, the brightness, time on and color is controlled and randomized to provide hundreds of thousands of sequences to avoid a noticeable pattern which would detract from the user's experience while using the device. It is possible to program each microprocessor uniquely so that no two devices have the exact same patterns. Also, since the interplay of the diffractor and diffusion is important, it is possible to vary the unique diffraction pattern on each device to provide a unique operation of each device so that no two devices share exactly the same patterns. Additionally, the placement of the LEDs in the base may be varied to further enhance the uniqueness of each device.

Additionally, the device may be used as a night light for those users who desire it to help get a restful night's sleep especially if the user is a child. In this case, the light would be set to have a fixed sequence to enhance its ability to function as a night light. Of course, it could also function as a night light for any age but it may be desirable to have the light operate with a more complicated sequence for older users. Also, in the embodiment that is used with children, it may be desirable to have the light diffractor made of plastic or some other break resistant material.

Although the instant invention has been described in relation to particular embodiments thereof, many other variations and modifications and other uses will become apparent to those skilled in the art.

What is claimed is:

1. An electronic stress relieving device comprising:
a base;
at least one light source disposed on said base wherein said at least one light source produces a light;
a light diffuser;
said light diffuser being at least partially translucent;
said light diffuser disposed over said at least one light source wherein said light is diffused when viewed through said light diffuser;
an energy source electrically connected to said at least one light source; and
an electronic controller in electrical communication with said at least one light source and said energy source wherein said at least one light source is controllable to operate said at least one light in a selected mode of operation.

2. The electronic stress relieving device according to claim 1 further comprising a light diffractor disposed to surround said light diffuser wherein said light is diffracted as said light passes through said light diffractor.

3. The electronic stress relieving device according to claim 2 further comprising:
a second light source;
a third light source; and
said second and third light source electrically connected to said energy source and said electronic controller.

4. The electronic stress relieving device according to claim 3 wherein said at least one light source, second light source and third light source are each selected to emit a different colored light.

5. The electronic stress relieving device according to claim 1 wherein said at least one light source is an LED light source.

6. The electronic stress relieving device according to claim 1 wherein said light diffuser is made of glass.

7. The electronic stress relieving device according to claim 1 wherein said light diffuser is made from plastic.

8. The electronic stress relieving device according to claim 1 wherein said electronic controller includes a microprocessor.

9. The electronic stress relieving device according to claim 1 wherein said energy source is a battery.

10. The electronic stress relieving device according to claim 8 further comprising at least one control switch electrically connected to said electronic controller wherein control signals are entered therein to control said selected mode of operation.

11. The electronic stress relieving device according to claim 9 further comprising a battery compartment whereby said battery is held in place.

12. The electronic stress relieving device according to claim 8 wherein said microprocessor provides a randomized mode of operation.

13. The electronic stress relieving device according to claim 8 wherein said microprocessor provides a selected time interval to energize said at least one light source.

14. The electronic stress relieving device according to claim 1 wherein said at least one light source is a multi-color light source.

15. The electronic stress relieving device according to claim 2 wherein said light diffractor is made of glass.

16. The electronic stress relieving device according to claim 2 wherein said light diffractor is made from plastic.

17. An electronic stress relieving device comprising:
a base;
at least one light source disposed on said base wherein said at least one light source produces a light;
a light diffractor;
said light diffractor disposed over said at least one light source wherein said light is diffracted when viewed through said light diffractor:
an energy source electrically connected to said at east one light source; and
an electronic controller in electrical communication with said at least one light source and said energy source wherein said at least one light source is controllable to operate said at least one light in a selected mode of operation.

18. The electronic stress relieving device according to claim 17 wherein said light diffractor is made of glass.

19. The electronic stress relieving device according to claim 17 wherein said light diffractor is made from plastic.

20. The electronic stress relieving device according to claim 17 wherein said at least one light source is an LED.
